(19) United States
(12) Patent Application Publication
(10) Pub. No.: US 2024/0108566 A1
(43) Pub. Date: Apr. 4, 2024

(54) COMPOSITION CONTAINING RECOMBINANT COLLAGEN WITH REPAIRING AND SOOTHING EFFECTS, EYE CREAM CONTAINING THE COMPOSITION AND PREPARATION METHOD THEREOR

(71) Applicant: MAYINGLONG MASSIVE HEALTH CO., LTD, Wuhan (CN)

(72) Inventors: Daonian ZHOU, Wuhan (CN); Chang TAN, Wuhan (CN); Jinlong SUN, Wuhan (CN); Mingjia LI, Wuhan (CN); Ge CHEN, Wuhan (CN); Weiping ZHANG, Wuhan (CN); Heng ZHANG, Wuhan (CN); Lei JIANG, Wuhan (CN); Li ZHOU, Wuhan (CN); Jincheng QI, Wuhan (CN)

(21) Appl. No.: 18/538,047

(22) Filed: Dec. 13, 2023

Related U.S. Application Data
(63) Continuation of application No. PCT/CN2023/108883, filed on Jul. 24, 2023.

(30) Foreign Application Priority Data
Aug. 30, 2022 (CN) 202211058014X

(51) Int. Cl. A61K 8/65; A61Q 19/00

(52) U.S. Cl. CPC A61K 8/65; A61Q 19/00; A61K 2800/48; A61K 2800/51; A61K 2800/522; A61K 2800/524; A61K 2800/80

(57) ABSTRACT

A composition containing recombinant collagen with repairing and soothing effects, eye cream containing the composition and preparation method thereof are provided. The composition contains the following raw materials in parts by weight: 1 to 10 parts of recombinant collagen, 1 to 10 parts of sodium hyaluronate, 1 to 5 parts of bifida ferment lysate, 1 to 15 parts of glucosylglycerol, 1 to 10 parts of bisabolol, 1 to 15 parts eight-treasure essence stock solution, and 1 to 15 parts of pink red plum rechecking essence. The composition features with good skin permeability, contributing to its significant effects on repairing skin barriers and relieving skin inflammation, as well as its advantages of low irritation and high safety. Thus, the composition is suitable for people with damaged eye skin and has good market application prospects.

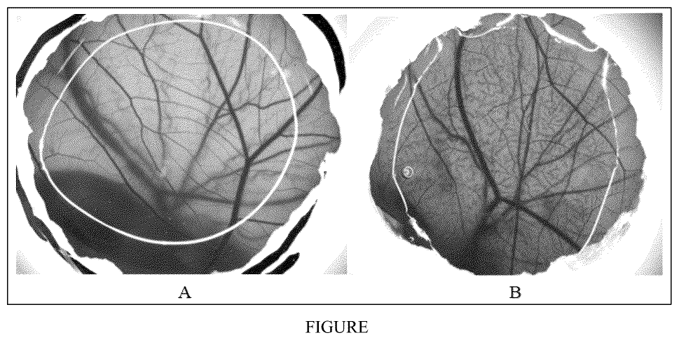

### TECHNICAL FIELD

The present disclosure relates to a composition containing recombinant collagen with repairing and soothing effects, eye cream containing the composition and preparation method thereof.

### BACKGROUND

The skin around the eyes is the weakest part of the human skin, and the thickness of its epidermis and dermis is far less than that of the facial skin, making it susceptible to external damage. With the growth of age and the increase of life pressure, factors such as staying up late, facing the computer for a long time, sunlight in the environment, artificial light exposure, air pollution, dust, particles and other factors can lead to accelerated aging of the skin around the eyes, causing skin damage, damaging the skin barrier around the eyes, leading to water loss of the skin around the eyes; thus, external stimuli can easily penetrate and damage the skin around the eyes, and can easily cause sensitive skin symptoms such as itching, tingling, burning, and tightness. It is widely believed that the occurrence of these symptoms is a complex process involving skin barrier-neurovascular-immune inflammation. Therefore, solving the above-mentioned sensitive and damaged problem of eye skin can be approached from two main aspects: repairing the skin barrier and soothing the skin inflammatory response.

At present, the common products targeting sensitive skin in the market mainly focus on soothing skin inflammation or repairing skin barriers as their functions, and rarely considered comprehensively based on the mentioned two aspects, resulting in unsatisfactory results. For example, patent application document CN114272179A discloses a composition for skin repair, which includes the following raw materials: carbomer, xanthan gum, sodium hyaluronate, glycerol, ethoxylated hydrogenated castor oil, recombinant collagen, licorice root extract, Centella asiatica extract, 1,2-hexanediol, p-hydroxyacetophenone, phenoxyethanol, methyl chloroformate, and the rest is deionized water; it utilizes the moisturizing and repairing effects of recombinant collagen, the anti-inflammatory and anti-allergic effects of licorice root extract, the tightening and repairing effects of Centella asiatica extract, and the anti-inflammatory and repairing effects of methyl chloroformate, contributing to strong repairing effects under the synergistic combination of various skin repair materials.

However, recombinant collagen, as a macromolecular protein, which is the main active ingredient in the above composition for skin repair, can only reach the epidermal layer of the skin when applied externally, and cannot reach the dermis through the skin matrix, leading to its poor skin permeability and limited application effect in functional dressings and skincare products. Thus, it is indicated that currently used functional dressings and skincare products containing recombinant collagen commonly have defects such as poor skin permeability and unclear application effects.

Therefore, improving the skin permeability and application efficiency of macromolecular proteins such as recombinant collagen is an urgent technical challenge for technical personnel in the field.

### SUMMARY

To address the short comings in the related art, the purpose of the present disclosure is to provide a composition containing recombinant collagen with repairing and soothing effects. The composition features with good skin permeability, contributing to its significant effects on repairing skin barriers and relieving skin inflammation, as well as its advantages of low irritation and high safety.

The another purpose of the present disclosure is to provide an eye cream containing the composition mentioned above, which features with high stability and low irritation.

The last purpose of the present disclosure is to provide a method for preparing the eye cream described above, and the method is simple and efficient, which is suitable for industrial production applications.

The technical solutions of the present disclosure are described as follows.

Firstly, the present disclosure provides a composition containing recombinant collagen protein with repairing and soothing effects, including the following preparation materials in parts by weight: 1 to 10 parts of recombinant collagen, 1 to 10 parts of sodium hyaluronate, 1 to 5 parts of bifida ferment lysate, 1 to 15 parts of glucosylglycerol, 1 to 10 parts of bisabolol, 1 to 15 parts of eight-treasure essence stock solution, 1 to 15 parts of pink red plum rechecking essence.

In the above technical solution, the recombinant collagen serves as an extracellular matrix to regulate cell growth, differentiation, migration, and metabolism, to promote cell growth, to accelerate wound repair, to inhibit scab formation. Additionally, the collagen peptide in recombinant collagen can form a thin film on the skin surface, which has good skin barrier effect. Therefore, recombinant collagen has a good repairing and barrier effect on damaged skin.

The sodium hyaluronate is not only a good moisturizer, but also has the effect of promoting the proliferation and differentiation of epidermal cells and clearing oxygen free radicals. It can accelerate the regeneration of epidermal cells, promote the repair of damaged skin, and make the skin delicate, smooth, and elastic.

The bifida ferment lysate can promote DNA damage and repair, prevent the formation of double strand breaks (micronuclei), and can also effectively protect the skin, maintain its vitality, reduce the risk of chronic light damage, which contributes to its significant positive effect on the repair of damaged skin. The bifida ferment lysate also has excellent anti-inflammatory and soothing effects, can alleviate skin dryness and sensitivity, alleviate stress states such as redness and allergies, and enhance the resistance of skin barriers, especially to environmental changes.

The glucosylglycerol is a glycosidic compound composed of glucose and glycerol linked by glycosidic bonds. It can penetrate deep into the dermis and has long-lasting moisturizing, repairing, and soothing effects. Its polyhydroxy structure can maintain cellular moisture and promote the synthesis of water channel proteins at the cellular level to provide comprehensive hydration for cells, resulting in good moisturizing effects. The glucosylglycerol can also promote the expression of type I collagen, thereby enhancing skin elasticity and collagen, and promoting skin barrier repair. In addition, The glucosylglycerol can significantly reduce the release of inflammatory factors, such as IL-1β, IL-1α, resulting in cell healing rate improvement and good anti-inflammatory and soothing effects.

The bisabolol is extracted from chrysanthemum plants and has significant anti-inflammatory and antibacterial effects. It serves as an active ingredient to protect allergic skin, reduce skin inflammation, and improve skin's anti irritation ability. It has the functions of repairing skin barriers, soothing skin inflammation, alleviating irritation, and anti-allergy. The bisabolol can also inhibit the enzymes responsible for collagen breakdown in the skin, prevent collagen breakdown, and stimulate collagen production.

The key component in the pink red plum rechecking essence is manuka, which belongs to Myrtaceae Juss and is also known as leptospermum scopa rium. The manuka is mainly wild and has bright flowers, and it also has rich essential oil, such as sesquiterpenes, sesquiterpenols, and sesquiterpenones. Modern pharmacological experiments have shown that the compound extract of the pink red plum rechecking essence can inhibit the production of TRPV1 and NO, inhibit the histamine signaling pathway, and have a strong inhibitory effect on local skin pain, erythema, and capillary dilation caused by external stimuli and inflammation through the synergistic effect of manuka, Stephania tetrandra, lotus, ect. Therefore, the pink red plum rechecking essence has good anti-inflammatory and soothing effects.

The eight-treasure essence stock solution includes Ligusticum chuanxiong root extract, Poria cocos sclerotium extract, atractylodes extract, Bletilla striata extract, Cayratia japonica root extract, artificial bezoar, pearl extract, and borneol. In the substances mentioned above, bezoar has antioxidant function; borneol has the effect of anti-corrosion and can promote muscle growth; pearl extract has the effect of brightening the eyes, calming the nerves, calming the wind, and regulating the skin and sores. The combination of these eight substances has good antioxidation, as well as good blood activating and stasis resolving effects, effectively preventing cell damage and repairing the skin barrier.

Specifically, the eight-treasure essence stock solution includes: 0.8 wt % Ligusticum chuanxiong root extract, 0.5 wt % Poria cocos sclerotium extract, 0.5 wt % Atractylodes macrocephala extract, 0.3 wt % Bletilla striata extract, 0.3 wt % Ampelopsis japonica root extract, 0.05 wt % artificial bezoar, 1 wt % pearl extract, and 0.05 wt % borneol, 1 wt % 1,2-hexanediol, 5 wt % butanediol, 0.5 wt % phenoxyethanol, 0.1 wt % ethyl hexyl glycerol, 89.9 wt % water. And, the eight-treasure essence stock solution can be obtained after uniformly mixing, filtering and sterilizating of the aforementioned raw materials.

In an embodiment, the composition containing recombinant collagen with repairing and soothing effects includes the following raw materials in parts by weight: 1 to 5 parts of recombinant collagen, 1 to 5 parts of sodium hyaluronate, 1 to 3 parts of bifida ferment lysate, 5 to 10 parts of glucosylglycerol, 5 to 10 parts of bisabolol, 5 to 10 parts of eight-treasure essence stock solution, 5 to 10 parts of pink red plum rechecking essence.

Secondly, the present disclosure provides an eye cream prepared from the aforementioned composition and auxiliary materials. Namely, in order to improve sensory properties, provide skin nutrition, and prevent quality deterioration, various ingredients commonly used in eye cosmetics can be appropriately added to the composition of the present disclosure as needed without damaging the effectiveness of the present disclosure.

In an embodiment, auxiliary materials are selected from one or more selected from the group consisting of polyol, moisturizing agent, moisturizer, emulsifier, thickener, antioxidant, chelating agent, preservative, pH regulator and deionized water.

In an embodiment, the emulsifier is a mixture of cetyl palmitate/sorbitan palmitate/sorbitan olivate and cetearyl olivate/sorbitan olivate; the moisturizer is one or more selected from the group consisting of Butyrospermum parkii (shea) butter, phytosteryl isostearate, poly(l-decene), isononyl isononanoate, poly(dimethylsiloxane), cetearyl alcohol, isocetane and decamethylcyclopentasiloxane; the thickener is one or more selected from the group consisting of pentaerythritol distearate, hydroxyethyl acrylate/sodium acryloyldimethyl taurate copolymer, cetearyl alcohol and beeswax. Specially, the composite ratio of cetyl palmitate/sorbitan palmitate/sorbitan olivate and cetearyl olivate/sorbitan olivate is 1:1 to 3, preferably 1:2.

In an embodiment, the chelating agent is one or more selected from the group consisting of ethylenediaminetetraacetic acid disodium salt (EDTA-2Na, also referred to as di sodium EDTA), ethylene diamine tetraacetic acid (EDTA) and sodium edetate (EDTA-4Na); the polyol is one or more selected from the group consisting of glycerol, propylene glycol, butanediol, sorbitol, dipropylene glycol, 1,3-propanediol, pentanediol and hexanediol; the antioxidant is one or more selected from the group consisting of tocotrienols, butylated hydroxytoluene, butylated hydroxyanisole and gallate esters; the preservative is one or more selected from the group consisting of phenoxyethanol, ethyl hexyl glycerol, chlorphenesin, methylparaben, p-hydroxyacetophenone, propylparaben, 1,2-hexanediol and sorbitan caprylate.

Thirdly, the present disclosure also provides a preparation method for the above-mentioned eye cream, including the following steps:
(1) the part of polyol, chelating agent, sodium hyaluronate and deionized water are mixed and then heated to evenly dispersed to obtain mixture I;
(2) the moisturizer, moisturizing agent, emulsifier, part of thickener, antioxidant, bisabolol and preservative are evenly heated and then added to the mixture I in step (1), followed by homogenization treatment and stir to obtain mixture II;
(3) the remained thickener and remained polyol are added to the mixture II in step (2) after thoroughly mixing, followed by heating and homogenization treatment, and mixture III is obtained after cooling;
(4) the recombinant collagen, glucosylglycerol, eight-treasure essence stock solution, pink red plum rechecking essence and bifida ferment lysate are added to the mixture III in step (3) after thoroughly mixing, followed by evenly dispersed, and mixture IV is obtained after cooling;
(5) the remained preservative is added to the mixture IV in step (4), followed by thoroughly mixing, homogenization treatment and vacuumization treatment, and the eye cream is obtained after cooling.

In an embodiment, the temperature of heating is 75-90° C. in step (1) and step (2); the mixture III is obtained when cooled to 40-50° C. in step (3); the mixture IV is obtained when cooled to 35-45° C. in step (4).

In an embodiment, the homogenization treatment is conducted under 1000-6000 rpm in step (2) and step (3).

Fourthly, the present disclosure also provides a method of eye care (i.e., application method of the above-mentioned composition), including: using the above-mentioned composition to prepare an eye care product. It can be understood that the preparation of the aforementioned eye cream is only a form of application, and the eye care product can also be eye essence, eye mask or another product.

The present disclosure has the following advantages:
(1) The composition containing recombinant collagen provided by the present disclosure is preferably composed of recombinant collagen, sodium hyaluronate, bifida ferment lysate, glucosylglycerol, bisabolol, eight-treasure essence stock solution and pink red plum rechecking essence. And the combination features with good skin permeability, contributing to its significant effects on repairing skin barriers and relieving skin inflammation, as well as its advantages of low irritation and high safety.
(2) The present disclosure utilizes glucosylglycerol as a natural cell activator that can penetrate into the dermis of the skin. It can not only help improve the skin permeability of macromolecular compounds such as recombinant collagen and sodium hyaluronate, but also promote the expression of collagen in the body. Bisabolol is used to inhibit the enzymes responsible for collagen breakdown in the skin, prevent collagen breakdown, and stimulate collagen production. The extra addition of bifida ferment lysate, pink red plum rechecking essence and eight-treasure essence stock solution contributes to enhancement of the repairing and soothing effect of the composition through the synergistic effect of multiple components.
(3) The bifida ferment lysate used in present disclosure, which is composed of metabolites, cytoplasmic fragments, cell wall components and polysaccharide complexes, is obtained by culturing, inactivating, and decomposing bifidobacteria. The processing of bifidobacteria produces abundant vitamins, minerals, amino acids, and kojic acid; but, the kojic acid can increase skin irritation, easily leading to side effects such as redness and pain on the skin, and is generally not suitable for sensitive muscle use. In the present disclosure, the composition described utilizes the synergy of various functional components and regulates the dosage of each component, to reduce the irritation of the composition, making it very suitable for use in sensitive muscles with skin damage.
(4) The present disclosure uses a mixture of cetyl palmitate/sorbitan palmitate/sorbitan olivate and cetearyl olivate/sorbitan olivate as the emulsifier, which synergistically improves the stability of the product with other components. Thus, the prepared product has a delicate texture, low particles when applied. In the meanwhile, it has no obvious discoloration or odor when stored at room temperature or under high and low temperature cycling conditions, and the paste has no layering phenomenon such as water or oil, making it can be stored for a long time without spoilage.
(5) The eye cream described in the present disclosure has a simple and efficient preparation method, and is suitable for industrial production and application.

### BRIEF DESCRIPTION OF DRAWING

FIGURE illustrates a comparison diagram of the chicken embryo stimulation in Embodiment 1 and Control group 1 during chick embryo chorioallantoic membrane test, where A represents Embodiment 1 without stimulation and B represents the Control group 1 with medium stimulation.

### DETAILED DESCRIPTION OF EMBODIMENTS

In the following, the technical solutions in the embodiments of the present disclosure will be clearly and completely described in combination with the embodiments of the present disclosure. Apparently, the described embodiments are only some of the embodiments of the present disclosure, but not the whole embodiments. Based on the embodiments in the present disclosure, all other embodiments obtained by those skilled in the art without creative labor belong to the scope of protection of the present disclosure.

It is noted that some substance information in the present disclosure is as follows:

The recombinant collagen was purchased from Jiangsu Jiangshan Juyuan Biotechnology Co., Ltd, China; the eight-treasure essence stock solution was self-produced by Ma Yinglong Health Co., Ltd, China; the pink red plum rechecking essence was purchased from Guangzhou Jianeng Biotechnology Co., Ltd, China; the trade name of cetearyl olivate/sorbitan olivate is Olivem®1000; the trade name of hydroxyethyl acrylate/sodium acryloyldimethyl taurate copolymer is SEPINOV™ WEO; the trade name of cetyl palmitate/sorbitan palmitate/sorbitan olivate is Oliwax® LC; and the trade name of phenoxyethanol/ethyl hexyl glycerol is Euxyl® PE 9010.

### Embodiment 1

A composition containing recombinant collagen protein with repairing and soothing effects, including the following preparation materials by weight: 2 g of recombinant collagen, 3 g of sodium hyaluronate, 2 g of bifida ferment lysate, 8 g of glucosylglycerol, 6 g of bisabolol, 6 g of eight-treasure essence stock solution, and 7 g of pink red plum rechecking essence.

Furthermore, in this embodiment, a preparation method for the aforementioned composition is provided, which includes: the above bisabolol is dissolved in 25 g of butanediol, and then 45 g of deionized water and other materials are added for mixing to obtain the composition.

### Embodiment 2

A composition containing recombinant collagen protein with repairing and soothing effects, including the following preparation materials by weight: 2 g of recombinant collagen, 2 g of sodium hyaluronate, 2 g of bifida ferment lysate, 6 g of glucosylglycerol, 5 g of bisabolol, 6 g of eight-treasure essence stock solution, and 10 g of pink red plum rechecking essence.

Furthermore, in this embodiment, a preparation method for the aforementioned composition is provided, which includes: the above bisabolol is dissolved in 20 g of butanediol, and then 50 g of deionized water and other materials are added for mixing to obtain the composition.

### Embodiment 3

A composition containing recombinant collagen protein with repairing and soothing effects, including the following preparation materials by weight: 3 g of recombinant collagen, 3 g of sodium hyaluronate, 1 g of bifida ferment lysate, 7 g of glucosylglycerol, 7 g of bisabolol, 8 g of eight-treasure essence stock solution, and 8 g of pink red plum rechecking essence.

Furthermore, in this embodiment, a preparation method for the aforementioned composition is provided, which includes: the above bisabolol is dissolved in 30 g of butanediol, and then 35 g of deionized water and other materials are added for mixing to obtain the composition.

### Embodiment 4

An eye cream with repairing and soothing effects, prepared by weight from the following raw materials:
Part A: 41.6 g of deionized water, 0.05 g of disodium EDTA, 3 g of glycerol, 2 g of sodium hyaluronate;
Part B: 2 g of poly(dimethylsiloxane), 3 g of cetearyl olivate/sorbitan olivate, 1.5 g of cetyl palmitate/sorbitan palmitate/sorbitan olivate, 1.5 g of pentaerythritol distearate, 2 g of isononyl isononanoate, 1 g of tocopheryl acetate, 6 g of bisabolol, 2 g of Butyrospermum parkii (shea) butter, 3 g of polysynlane, 0.8 g of sorbitan caprylate;
Part C: 0.6 g of hydroxyethyl acrylate/sodium acryloyldimethyl taurate copolymer, 4 g of butanediol;
Part D: 2 g of recombinant collagen, 8 g of glucosylglycerol, 7 g of pink red plum rechecking essence, 6 g of eight-treasure essence stock solution, 2 g of bifida ferment lysate;
Part E: 0.35 g of phenoxyethanol/ethyl hexyl glycerol, 0.6 g of 1,2-hexanediol.

Furthermore, in this embodiment, a preparation method for the aforementioned eye cream is provided, which includes the following steps.

In step (1), the materials in Part A are added to the emulsification pot, and then are heated to 80° C. until evenly dispersed to obtain mixture I.

In step (2), the materials in Part B are added to the oil bath, and then heated to 85° C. and stirred until evenly dispersed, and then are added to the mixture I in step (1) at a uniform rate; afterwards, the materials are first homogenized and emulsified at a speed of 3000 rpm for 3 min, and then stirred at a speed of 400 rpm to obtain mixture II.

In step (3), the materials in Part C are added to the mixture II in step (2) after mixing, and then are homogenized and emulsified at a speed of 3000 rpm for 10 min, followed by cooling to 45° C., to obtain mixture III.

In step (4), the materials in Part D are added to the mixture III in step (3) after mixing, and then are evenly dispersed before cooling to 40° C., to obtain mixture IV.

In step (5), the materials in Part E are added to the mixture IV in step (4) after mixing, followed by thoroughly mixing, homogenization treatment and vacuumization treatment, and the eye cream is obtained after cooling to 35° C. and then to room temperature.

### Embodiment 5

An eye cream with repairing and soothing effects, prepared by weight from the following raw materials:
Part A: 38.4 g of deionized water, 0.05 g of disodium EDTA, 5 g of glycerol, 3 g of sodium hyaluronate;
Part B: 2 g of poly(dimethylsiloxane), 2 g of cetearyl olivate/sorbitan olivate, 1 g of cetyl palmitate/sorbitan palmitate/sorbitan olivate, 2 g of pentaerythritol distearate, 2 g of isononyl isononanoate, 1 g of tocopheryl acetate, 5 g of bisabolol, 2 g of Butyrospermum parkii (shea) butter, 3 g of polysynlane, 1 g of sorbitan caprylate;
Part C: 0.6 g of hydroxyethyl acrylate/sodium acryloyldimethyl taurate copolymer, 6 g of butanediol;
Part D: 2 g of recombinant collagen, 6 g of glucosylglycerol, 8 g of pink red plum rechecking essence, 7 g of eight-treasure essence stock solution, 2 g of bifida ferment lysate;
Part E: 0.35 g of phenoxyethanol/ethyl hexyl glycerol, 0.6 g of 1,2-hexanediol.

Furthermore, in this embodiment, a preparation method for the aforementioned eye cream is provided, which includes the following steps.

In step (1), the materials in Part A are added to the emulsification pot, and then are heated to 80° C. until evenly dispersed to obtain mixture I.

In step (2), the materials in Part B are added to the oil bath, and then are heated to 85° C. and stirred until evenly dispersed, and then are added to the mixture I in step (1) at a uniform rate; afterwards, the materials are first homogenized and emulsified at a speed of 3000 rpm for 3 min, and then stirred at a speed of 400 rpm to obtain mixture II.

In step (3), the materials in Part C are added to the mixture II in step (2) after mixing, and then are homogenized and emulsified at a speed of 3000 rpm for 10 min before cooling to 45° C., to obtain mixture III.

In step (4), the materials in Part D are added to the mixture III in step (3) after mixing, and then are evenly dispersed before cooling to 40° C., to obtain mixture IV.

In step (5), the materials in Part E are added to the mixture IV in step (4) after mixing, followed by thoroughly mixing, homogenization treatment and vacuumization treatment, and the eye cream is obtained after cooling to 35° C. and then to room temperature.

### Embodiment 6

An eye cream with repairing and soothing effects, prepared by weight from the following raw materials:
Part A: 38.7 g of deionized water, 0.05 g of disodium EDTA, 4 g of glycerol, 1 g of sodium hyaluronate;
Part B: 2 g of poly(dimethylsiloxane), 4 g of cetearyl olivate/sorbitan olivate, 2 g of cetyl palmitate/sorbitan palmitate/sorbitan olivate, 1 g of pentaerythritol distearate, 2 g of isononyl isononanoate, 1 g of tocopheryl acetate, 7 g of bisabolol, 2 g of Butyrospermum parkii (shea) butter, 3 g of polysynlane, 0.7 g of sorbitan caprylate;
Part C: 0.6 g of hydroxyethyl acrylate/sodium acryloyldimethyl taurate copolymer, 5 g of butanediol;
Part D: 2 g of recombinant collagen, 10 g of glucosylglycerol, 6 g of pink red plum rechecking essence, 5 g of eight-treasure essence stock solution, 2 g of bifida ferment lysate;
Part E: 0.35 g of phenoxyethanol/ethyl hexyl glycerol, 0.6 g of 1,2-hexanediol.

Furthermore, in this embodiment, a preparation method for the aforementioned eye cream is provided, which includes the following steps.

In step (1), the materials in Part A are added to the emulsification pot, and then are heated to 80° C. until evenly dispersed to obtain mixture I.

In step (2), the materials in Part B are added to the oil bath, and then are heated to 85° C. and stirred until evenly disperse, and then are added to the mixture I in step (1) at a uniform rate; afterwards, the materials are first homogenized and emulsified at a speed of 3000 rpm for 3 min, and then stirred at a speed of 400 rpm to obtain mixture II.

In step (3), the materials in Part C are added to the mixture II in step (2) after mixing, and then are homogenized and emulsified at a speed of 3000 rpm for 10 min before cooling to 45° C., to obtain mixture III.

In step (4), the materials in Part D are added to the mixture III in step (3) after mixing, and then are evenly dispersed before cooling to 40° C., to obtain mixture IV.

In step (5), the materials in Part E are added to the mixture IV in step (4) after mixing, followed by thoroughly mixing, homogenization treatment and vacuumization treatment, and the eye cream is obtained after cooling to 35° C. and then to room temperature.

### Control Group 1

A single compound, which is bifida ferment lysate, is provided.

### Control Group 2

A composition containing recombinant collagen protein with repairing and soothing effects, including the following preparation materials by weight: 2 g of recombinant collagen, 3 g of sodium hyaluronate, 6 g of bifida ferment lysate, 8 g of glucosylglycerol, 6 g of bisabolol, 8 g of eight-treasure essence stock solution, 7 g of pink red plum rechecking essence.

Furthermore, in this Control group, a preparation method for the aforementioned composition is provided, which includes: the above bisabolol is dissolved in 25 g of butanediol, and then 45 g of deionized water and other materials are added for mixing to obtain the composition.

### Control Group 3

A composition containing recombinant collagen protein with repairing and soothing effects, including the following preparation materials by weight: 2 g of recombinant collagen, 2 g of sodium hyaluronate, 8 g of bifida ferment lysate, 6 g of glucosylglycerol, 5 g of bisabolol, 6 g of eight-treasure essence stock solution, 10 g of pink red plum rechecking essence.

Furthermore, in this Control group, a preparation method for the aforementioned composition is provided, which includes: the above bisabolol is dissolved in 20 g of butanediol, and then 50 g of deionized water and other materials are added for mixing to obtain the composition.

### Control Group 4

Compared with Embodiment 4, the difference lies only in the absence of cetyl palmitate/sorbitan palmitate/sorbitan olive oleate in Part B of Control group 4, and the other components and preparation methods are consistent.

### Control Group 5

Compared with Embodiment 4, the difference lies only in the absence of glucosylglycerol in Part D of Control group 5, and the other components and preparation methods are consistent.

### Control Group 6

Compared with Embodiment 4, the difference lies only in the absence of bisabolol in Part B of Control group 4, and the other components and preparation methods are consistent.

### Experiment 1

The chicken embryo chorioallantoic membrane test (HET CAM Test) is a test method certified by the European Center for Alternative Methods (ECVAM) to test the irritation of products. It is currently one of the main research methods for in vitro eye irritation testing of cosmetics and chemicals, and can be used to evaluate the irritation of various substances. Thus, eye irritation tests in vitro on the prepared compositions were performed.

Unlike experiments conducted solely on cell lines, the chicken embryo allantoic membrane can be regarded as a complete organism for the rich blood vessels of its surface, which makes it of high potential as an alternative method for eye stimulation.

The basic principle of this method is that the chorioallantoic membrane (CAM) of chicken embryos, which is close to the eggshell membrane and has abundant blood vessels, is the respiratory membrane of chicken embryos, and making it can be seen as a system with abundant blood vessels but no perception. By utilizing the similar characteristics of CAM and rabbit conjunctiva structure, the CAM of chicken embryos can be used as a screening method in eye irritation by observing the changes in blood vessels after CAM exposure (bleeding, hemolysis, coagulation) and detecting the damage of chemicals to CAM to evaluate the potential irritation of chemicals. Therefore, the HET-CAM test has been internationally validated as an alternative method to the eye irritation Draize test.

The specific description of the test method is as follows:
(1) Egg selection: SPF grade white Laihang chicken fertilized eggs (purchased by Jinan Saisi Poultry Technology Co., Ltd.) are chosen. The egg quality meets the requirements of relevant standards, and the supplier should have the qualification of “SPF Chicken (Egg) Designated Production Enterprise for Veterinary Medicine Production and Inspection” recognized by the agricultural department. Eggs should be fresh, clean, and intact, with a weight of 50 g to 60 g. When hatching to the age of 4 days, eggs should be examined and unfertilized chicken embryos should be discarded. Broken or thin shell chicken embryos cannot be used.
(2) Preparation of experimental chicken embryos: SPF chicken embryos are purchased and placed in an incubator with the air chamber facing upwards. The incubation temperature is 37.5° C.±0.5° C., relative humidity is 60%±5%, and the turntable frequency is once/30 minutes. When incubated until the age of 9 days, the hatching condition is checked with an egg incubator, and unfertilized, inactive, defective, deformed, broken or thin shell chicken embryos are not allowed to be used.
(3) Preparation of allantoic membrane (CAM): 9-day-old chicken embryos were examined by egg photography and the position of the air chamber was marked; subsequently, tweezers are used to remove the eggshell part from the air chamber, and 0.9% sodium chloride (NaCl) solution is added dropwise to moisten the inner membrane, and then tweezers are used to remove it without damaging blood vessels; otherwise, it should be discarded when damaged.
(4) Formal experiment: the test method is selected based on the characteristics of the test substance, and if the test substance is transparent liquid, reaction time method is chosen. Specifically, the test substance in the test is a transparent liquid, so the reaction time method is chosen. During specific operations, 0.3 mL of the test substance is taken and directly dropped onto the CAM surface for 5 minutes, and then observing and recording the time (s) when bleeding, coagulation, and vascular fusion occur. Repeat for 6 chicken embryos.

Test sample: the compositions prepared in Embodiments 1-3 and Control groups 1-3.

Test results: the evaluation of the stimulation scoring method for the chicken embryo chorioallantoic membrane test is shown in Table 1, and the stimulation results of the compositions prepared in the Embodiments and in Control groups are shown in Table 2.

TABLE 1
Method for evaluating the results of stimulation
scoring method

| stimulation score | stimulation classification |
|---|---|
| IS < 1 | No stimulation |
| 1 ≤ IS ≤ 5 | Low stimulation |
| 5 ≤ IS ≤ 9 | Moderate stimulation |
| IS ≥ 10 | Strong stimulation/corrosiveness |

TABLE 2
The stimulation results of the compositions prepared
in Embodiments and Control groups

| | Stimulus | Stimulus |
|---|---|---|
| No. | score (IS) | classification |
| Embodiment 1 | 0.6 | No stimulation |
| Embodiment 2 | 0.8 | No stimulation |
| Embodiment 3 | 0.5 | No stimulation |
| Control group 1 | 6 | Moderate stimulation |
| Control group 2 | 4 | Low/Moderate stimulation |
| Control group 3 | 5 | Low/Moderate stimulation |

From Table 2, it can be seen that the bifida ferment lysate tested separately for Control group 1 has moderate irritation to chicken embryos, which is not suitable for people with weak stratum corneum and sensitive muscle populations for the reason that bifid yeast has the effect of accelerating stratum corneum shedding and accelerating stratum corneum metabolism. Correspondingly, the chicken embryo stimulation scores of the compositions prepared in Embodiments 1-3 are all less than 1, showing no irritation, and it is due to the combination of bifida ferment lysate and other components. In the meanwhile, the chicken embryo stimulation scoring method of the prepared composition showed mild/moderate irritation with the dosage of the bifida ferment lysate in Control groups 2 and 3.

### Experiment 2

Stability performance tests are conducted on the eye creams prepared in Embodiment 4 and Control group 4, respectively, and the specific testing methods and results are as follows:

Test method: 12 transparent glass bottles (50 mL) were prepared and 40-50 g of the eye creams prepared in Embodiment 4 and Control group 4 were placed into the bottles, and then 10 bottles were placed at low temperature −7° C., low temperature 4° C., cycle −7° C./45° C./24 hours, high temperature 45° C., and room temperature 25° C. for 30 days, and the left two bottles were placed at high temperature 60° C. for 7 days.

Stability determination standard: the samples were compared with the samples under 4° C. after the samples were restored to room temperature. The sample could be considered stable when: 1) there was no obvious discoloration or odor during normal temperature storage or high and low temperature cycling; and 2) there was no particles or precipitates when applied, and 3) the sample had no stratification such as water or oil.

The specific results of stability testing are evaluated as shown in Table 3.

TABLE 3
Stability testing results of the eye creams in
Embodiment 4 and Control group 4

| | Embodiment 4 | Control group 4 |
|---|---|---|
| 4° C./30 days | normal | normal |
| 25° C./30 days | normal | normal |
| cycle/30 days | normal | particles and |
| | | precipitates occurred |
| −7° C./30 days | normal | normal |
| 45° C./30 days | normal | particles and |
| | | precipitates occurred |
| 60° C./7 days | normal | particles and |
| | | precipitates occurred |

The experimental results show that the product prepared in Embodiment 4 of the present disclosure has a delicate texture, and has no particles when applied; in the meanwhile, the product prepared in Embodiment 4 is easy to use, and has no obvious color change or taste change when stored at room temperature and circulated at high and low temperatures. The product also has no layering phenomenon such as water and oil, and there are no abnormalities in stability inspection. Thus, it is considered that the product has good stability. However, for product prepared in Control group 4, it seem fatty and its absorption is not fast enough. In addition, after high and low temperature cycling and high temperature environment, precipitates appear in the paste, and there is a granular feeling when applied, resulting in poor product stability.

### Experiment 3

An example of epidermal water loss (TEWL) test is used to evaluate the repair efficacy of eye creams prepared in Embodiments and Control groups. The instruments, equipment, testing methods, and test results used are as follows:

Instrument and equipment: skin surface moisture loss instrument, Tewameter® TM 300, Courage&Khazaka, Germany.

Method: 30 subjects aged 18-60 with dry and rough skin around the eyes and fragile skin barriers were randomly selected and divided into 3 groups of 10 people each. And then, the products in Embodiment 4 and Control group 5-6 are evenly applied to the skin around the eyes of each group of subjects for 10-15 minutes, followed by gently massage until the complete absorption of the product. The application is conducted once in the morning and once in the evening. During the entire testing, prolonged exposure to sunlight, outdoor activities, tourism, etc., and the use of cosmetics or drugs with similar efficacy to the product are not allowed; in the meanwhile, the change of the participants' daily care habits is not allowed.

Statistical method: each participant was tested three times to obtain the average value, which was then analyzed using SPSS software. The lower the TEWL value obtained, the less transdermal water loss.

Test results: as shown in Table 4.

TABLE 4
Results of transcutaneous water diversion loss
before and after the use of the product on the
skin of the subjects

| | TEWL [g/(h · m²)] | | |
|---|---|---|---|
| product | before | 14 days later | 28 days later |
| Embodiment 4 | 17.53 ± 2.49 | 14.12 ± 2.79** | 11.85 ± 2.18** |
| Control group 5 | 16.69 ± 2.51 | 14.68 ± 2.50 | 14.05 ± 2.24 |
| Control group 6 | 17.0 ± 63.44 | 15.37 ± 3.33 | 13.54 ± 3.34* |

Note:
compared with the results before the use of product, *P <0.05, **P <0.01

From Table 4, it can be seen that after 14 days of using the product in the Embodiment 4, the amount of transcutaneous water diversion loss around the eye showed a very significant decrease (p<0.01). After 28 days of using the product, the amount of transcutaneous water diversion loss further decreased. It shows that the product has good repair effect and takes effect quickly. Correspondingly, after 14 days of using the product in Control group 5 and Control group 6, there was no significant difference in the amount of transdermal water loss around the eyes. Therefore, it can be concluded that the repair effect of the product in Embodiment 4 on dry, rough, and fragile skin around the eyes is much better than that of the product in the Control groups.

### Experiment 4

The present disclosure refers to the standard “Cosmetic Soothing Efficacy Test-In Vitro TNF-α Measurement of Inflammatory Factor Content-Lipopolysaccharide Induced Macrophage RAW264.7 Test Method (T/SHRH 034-2020)” to determine the soothing effect of eye creams prepared in the Embodiments and Control groups.

Based on the principle that the bacterial lipopolysaccharides (LPS) induce macrophage line RAW264.7 to secrete inflammatory factor TNF-α, the test of the amount of the TNF-α after action of different test substances can be used to evaluate the inhibition of test substances on the secretion of TNF-α. Specifically, the evaluation indicators are TNF-α content and TNF-α inhibition rate, and TNF-α content is obtained by the calculating average value of the results of three tests via enzyme-linked immunosorbent assay (ELISA); and TNF-α inhibition rate is calculated by following formula:

TNF-α inhibition rate (%)=(1−average content of TNF-α in action of test group/mean content of TNF-α in negative control group)×100%.

First, the eye creams prepared in Embodiment 4, Control group 5, and Control group 6 are weighted separately, and then the eye cream is dissolved in the solvent dimethyl sulfoxide, shook and mixed thoroughly to obtain different dosage of 2000 μg/mL, which were used in test group a, test group b, and test group c for efficacy testing, respectively. In addition, the negative control group was given an equal amount of LPS containing cell culture medium, while the positive control group was given cell culture with an equal amount of 100 μg/mL dexamethasone and LPS. TNF-α Content was detected and TNF-α inhibition rate was calculated as shown in Table 5.

TABLE 5

| | TNF-a | TNF-a |
|---|---|---|
| Group | content (ng/L) | inhibition rate (%) |
| Negative control | 1959.6 ± 56.196 | — |
| group | | |
| Positive control | 969.8 ± 42.877** | 50.51% |
| group | | |
| Test group a | 1370.0 ± 55.019** | 30.09% |
| Test group b | 1671.6 ± 75.479** | 14.70% |
| Test group c | 1592.3 ± 60.483** | 18.74% |

Note: compared with negative control group, *P < 0.05, **P < 0.01

From Table 5, it can be seen that compared with the negative control group, the TNF-α content of the positive control group and the test group decreased significantly and had extremely significant differences (P<0.01), but by comparing TNF-α inhibition rate can be observed that the anti-inflammatory effect of test group a is significantly better than that of test group b and c, indicating that the eye cream in test group a has better anti-inflammatory and soothing effects on eye damaged skin.

The present disclosure illustrates the eye cream with repairing and soothing effects and its preparation method through the above embodiments, but the present disclosure is not limited to the above embodiments, which does not mean that the present disclosure must rely on the above embodiments to be implemented. Those skilled in the art should be aware that any improvements to the present disclosure, equivalent substitution of individual raw materials and addition of auxiliary components to the product of the present disclosure, and selection of specific methods fall within the scope of protection of the present disclosure.

What is claimed is:

1. A composition containing recombinant collagen with repairing and soothing effects, wherein the composition comprises the following raw materials in parts by weight: 1 to 10 parts of recombinant collagen, 1 to 10 parts of sodium hyaluronate, 1 to 5 parts of bifida ferment lysate, 1 to 15 parts of glucosylglycerol, 1 to 10 parts of bisabolol, 1 to 15 parts of eight-treasure essence stock solution, and 1 to 15 parts of pink red plum rechecking essence.

2. The composition as claimed in claim 1, wherein the composition comprises the following raw materials in parts by weight: 1 to 5 parts of the recombinant collagen, 1 to 5 parts of the sodium hyaluronate, 1 to 3 parts of the bifida ferment lysate, 5 to 10 parts of the glucosylglycerol, 5 to 10 parts of the bisabolol, 5 to 10 parts of the eight-treasure essence stock solution, and 5 to 10 parts of the pink red plum rechecking essence.

3. The composition as claimed in claim 1, wherein the composition comprises the following raw materials in parts by weight: 2 parts of the recombinant collagen, 3 parts of the sodium hyaluronate, 2 parts of the bifida ferment lysate, 8 parts of the glucosylglycerol, 6 parts of the bisabolol, 6 parts of the eight-treasure essence stock solution, and 7 parts of the pink red plum rechecking essence.

4. The composition as claimed in claim 1, wherein the composition comprises the following raw materials in parts by weight: 2 parts of the recombinant collagen, 2 parts of the sodium hyaluronate, 2 parts of the bifida ferment lysate, 6 parts of the glucosylglycerol, 5 parts of the bisabolol, 6 parts of the eight-treasure essence stock solution, and 10 parts of the pink red plum rechecking essence.

5. The composition as claimed in claim 1, wherein the composition comprises the following raw materials in parts by weight: 3 parts of the recombinant collagen, 3 parts of the sodium hyaluronate, 1 part of the bifida ferment lysate, 7 parts of the glucosylglycerol, 7 parts of the bisabolol, 8 parts of the eight-treasure essence stock solution, and 8 parts of the pink red plum rechecking essence.

6. An eye cream, wherein the eye cream is prepared by the composition as claimed in claim 1 and an auxiliary material.

7. The eye cream as claimed in claim 6, wherein the auxiliary material is one or more selected from the group consisting of polyol, moisturizing agent, moisturizer, emulsifier, thickener, antioxidant, chelating agent, preservative, pH regulator, and deionized water.

8. The eye cream as claimed in claim 7, wherein the emulsifier is a mixture of cetyl palmitate/sorbitan palmitate/sorbitan olivate and cetearyl olivate/sorbitan olivate; the moisturizer is one or more selected from the group consisting of Butyrospermum parkii (shea) butter, phytosteryl isostearate, poly(l-decene), isononyl isononanoate, poly(dimethylsiloxane), cetearyl alcohol, isocetane and decamethylcyclopentasiloxane; the thickener is one or more selected from the group consisting of pentaerythritol distearate, hydroxyethyl acrylate/sodium acryloyldimethyl taurate copolymer, cetearyl alcohol and beeswax.

9. The eye cream as claimed in claim 7, wherein the chelating agent is one or more selected from the group consisting of ethylenediaminetetraacetic acid disodium salt (EDTA-2Na), ethylene diamine tetraacetic acid (EDTA) and sodium edetate (EDTA-4Na); the polyol is one or more selected from the group consisting of glycerol, propylene glycol, butanediol, sorbitol, dipropylene glycol, 1,3-propanediol, pentanediol and hexanediol; the antioxidant is one or more selected from the group consisting of tocotrienols, butylated hydroxytoluene, butylated hydroxyanisole and gallate esters; the preservative is one or more selected from the group consisting of phenoxyethanol, ethyl hexyl glycerol, chlorphenesin, methylparaben, p-hydroxyacetophenone, propylparaben, 1,2-hexanediol and sorbitan caprylate.

10. The eye cream as claimed in claim 6, wherein the composition comprises the following raw materials in parts by weight: 1 to 5 parts of the recombinant collagen, 1 to 5 parts of the sodium hyaluronate, 1 to 3 parts of the bifida ferment lysate, 5 to 10 parts of the glucosylglycerol, 5 to 10 parts of the bisabolol, 5 to 10 parts of the eight-treasure essence stock solution, and 5 to 10 parts of the pink red plum rechecking essence.

11. The eye cream as claimed in claim 6, wherein the composition comprises the following raw materials in parts by weight: 2 parts of the recombinant collagen, 3 parts of the sodium hyaluronate, 2 parts of the bifida ferment lysate, 8 parts of the glucosylglycerol, 6 parts of the bisabolol, 6 parts of the eight-treasure essence stock solution, and 7 parts of the pink red plum rechecking essence.

12. The eye cream as claimed in claim 6, wherein the composition comprises the following raw materials in parts by weight: 2 parts of the recombinant collagen, 2 parts of the sodium hyaluronate, 2 parts of the bifida ferment lysate, 6 parts of the glucosylglycerol, 5 parts of the bisabolol, 6 parts of the eight-treasure essence stock solution, and 10 parts of the pink red plum rechecking essence.

13. The eye cream as claimed in claim 6, wherein the composition comprises the following raw materials in parts by weight: 3 parts of the recombinant collagen, 3 parts of the sodium hyaluronate, 1 part of the bifida ferment lysate, 7 parts of the glucosylglycerol, 7 parts of the bisabolol, 8 parts of the eight-treasure essence stock solution, and 8 parts of the pink red plum rechecking essence.

14. A method for preparing the eye cream as claimed in claim 7, comprising the following steps:
(1) mixing a part of the polyol, the chelating agent, the sodium hyaluronate and the deionized water, and then heating to make them evenly dispersed to obtain a mixture I;
(2) heating and stirring evenly the moisturizer, the moisturizing agent, the emulsifier, a part of the thickener, the antioxidant, the bisabolol and a part of the preservative, then adding them to the mixture I obtained in step (1), followed by homogenization treatment and stirring to obtain a mixture II;
(3) adding the remained thickener and the remained polyol to the mixture II obtained in step (2) after thoroughly mixing, followed by heating and homogenization treatment, and obtaining a mixture III after cooling;
(4) adding the recombinant collagen, the glucosylglycerol, the eight-treasure essence stock solution, the pink red plum rechecking essence and the bifida ferment lysate to the mixture III obtained in step (3) after thoroughly mixing, followed by evenly dispersed, and obtaining a mixture IV after cooling; and
(5) adding the remained preservative to the mixture IV obtained in step (4), followed by thoroughly mixing, homogenization treatment and vacuumization treatment, and obtaining the eye cream after cooling.

15. The method as claimed in claim 14, wherein a temperature of the heating is in a range of 75° C. to 90° C. in each of step (1) and step (2); the mixture III is obtained when cooled to 40-50° C. in step (3); and the mixture IV is obtained when cooled to 35-45° C. in step (4).

16. The method as claimed in claim 14, wherein the homogenization treatment is conducted under 1000 to 6000 rpm in each of step (2) and step (3).

17. The method as claimed in claim 14, wherein the emulsifier is a mixture of cetyl palmitate/sorbitan palmitate/sorbitan olivate and cetearyl olivate/sorbitan olivate; the moisturizer is one or more selected from the group consisting of Butyrospermum parkii (shea) butter, phytosteryl isostearate, poly(l-decene), isononyl isononanoate, poly(dimethylsiloxane), cetearyl alcohol, isocetane and decamethylcyclopentasiloxane; the thickener is one or more selected from the group consisting of pentaerythritol distearate, hydroxyethyl acrylate/sodium acryloyldimethyl taurate copolymer, cetearyl alcohol and beeswax.

18. The method as claimed in claim 14, wherein the chelating agent is one or more selected from the group consisting of ethylenediaminetetraacetic acid disodium salt (EDTA-2Na), ethylene diamine tetraacetic acid (EDTA) and sodium edetate (EDTA-4Na); the polyol is one or more selected from the group consisting of glycerol, propylene glycol, butanediol, sorbitol, di propyl ene glycol, 1,3-propanediol, pentanediol and hexanediol; the antioxidant is one or more selected from the group consisting of tocotrienols, butylated hydroxytoluene, butylated hydroxyanisole and gallate esters; the preservative is one or more selected from the group consisting of phenoxyethanol, ethyl hexyl glycerol, chlorphenesin, methylparaben, p-hydroxyacetophenone, propylparaben, 1,2-hexanediol and sorbitan caprylate.

19. The method as claimed in claim 14, wherein the composition comprises the following raw materials in parts by weight: 1 to 5 parts of the recombinant collagen, 1 to 5 parts of the sodium hyaluronate, 1 to 3 parts of the bifida ferment lysate, 5 to 10 parts of the glucosylglycerol, 5 to 10 parts of the bisabolol, 5 to 10 parts of the eight-treasure essence stock solution, and 5 to 10 parts of the pink red plum rechecking essence.

20. A method for applying the composition as claimed in claim 1, comprising: using the composition to prepare an eye care product.
